(19) United States
(12) Patent Application Publication
(10) Pub. No.: US 2010/0144544 A1
(43) Pub. Date: Jun. 10, 2010

(54) FLUORESCENCE LABELLING

(75) Inventors: George William Fraser, Leicester (GB); David J.M. Ray, Milton Keynes (GB)

Correspondence Address: LOGINOV & ASSOCIATES, PLLC, 10 WATER STREET, CONCORD, NH, 03301, US

(73) Assignee: UNIVERSITY OF LEICESTER, Leicester (UK)

(21) Appl. No.: 12/522,237

(22) PCT Filed: Jan. 4, 2008
(86) PCT No.: PCT/GB2008/050009

§ 371 Date: Nov. 30, 2009

(30) Foreign Application Priority Data
Jan. 5, 2007 (GB) 0700189.4

(51) Int. Cl. C40B 30/04; C40B 40/06; C40B 50/00; C40B 60/12; C07H 21/00

(52) U.S. Cl. 506/9; 506/16; 506/23; 506/39; 536/23.1

(57) ABSTRACT

Fluorescence Labelling This invention generally relates to techniques for fluorescence labelling, and to methods, apparatus and computer program code for processing fluorescence signal data. A method of determining respective first and second degree-of-labelling signals for different respective first and second fluorophores associated with a common entity, the method comprising: determining a first fluorescence signal from said first and second fluorophores under first conditions; determining a second fluorescence signal from said first and second fluorophores under second conditions different to said first conditions; and determining said first and second degree-of-labelling signals for said first and second fluorophores from said first and second fluorescence signals; and wherein said determining of said first and second degree-of-labelling signals is responsive to at least one coupling value (c₁₂; c₂₁) representing a coupling of energy between said fluorophores.

### FIELD OF THE INVENTION

This invention generally relates to techniques for fluorescence labelling, and to methods, apparatus and computer program code implementing numerical algorithms for processing fluorescence signal data. The techniques we describe are particularly useful in biotechnology applications.

### BACKGROUND TO THE INVENTION

A common biological problem is the measurement of the optical emission from spatially coincident fluorophores (dyes). Imaging the functional components of a living cell often involves the registration of multiple fluorescent markers. Quantifying the hybridisation of labelled nucleic acids (probes) to immobilised target molecules in a microarray (“gene chip”) can also require the simultaneous detection of multiple-component fluorescent spectra.

We have previously described, in WO 03/023376 (hereby incorporated by reference in its entirety) cryogenic detector technology, in particular employing a superconducting tunneling junction (STJ), for the detection of a fluorescent signal from, for example, a DNA (deoxyribonucleic acid) microarray. An STJ device is sensitive over a range of wavelengths (colours or energies), generally down to the single photon level, as well as exhibiting a highly linear response and high signal-to-noise ratio. The energy-resolving capability of the STJ in the optical band (embodiments of the device may be described as hyperspectral) facilitates simultaneous multi-colour detection of hybridisation to microarrays for applications such as drug discovery.

In a typical microarray experiment two samples or targets are reverse transcribed into cDNA (complementary DNA) and labelled using different fluorescent dyes. The DNA microarray comprises an array of DNA sequences which act as probes, and the targets are mixed and hybridised with these probes and then, after removal of excess unbound material by washing, the microarray is imaged, generally using a scanner which responds to the fluorescence signal at each of the array spots. The differential hybridisation of the two targets to a probe sequence is, broadly speaking, determined by the ratio of the fluorescence intensities at the spot on the microarray for the probe sequence. In this way, the relative abundance of each of the probe sequences in the two targets may be assessed. There are a number of variants of this basic technique. Currently the majority of microarrays comprise DNA (which here includes cDNA), but microarrays may also be fabricated using RNA (ribonucleic acid), proteins, antibodies, antigens and the like.

A conventional scanner typically employs a photomultiplier to record the signals from the microarray but we have described how an STJ detector device can be used to provide substantial improvements in performance (ibid; also Review of Scientific Instruments, Volume 74, Number 9, September 2003, “Detection of multiple fluorescent labels using superconducting tunnel junction detectors”, G. W. Fraser. J. S. Helsop-Harrison, T. Schwarzacher, A. D. Holland, P. Verhoeve and A. Peacock). For example one detector used in a study of biological fluorescence was a single 30×30 μm² STJ with 100 nm thick Ta layers and 30 nm thick Al layers on either side of the tunnel barrier. The detector was made using photolithographic techniques from a Ta/Al multilayer deposited on a polished sapphire substrate. Cooling to 300 mK in a ³He cryostat (i.e., T˜T_{c}/15, where T_{c} denotes the superconducting transition temperature) kept the thermally excited quasiparticle current well below the leakage current level. The STJ had a measured resolving power (λ/Δλ) of 14.1 at 600 nm. Samples were stimulated with a Leica microscope with mercury lamp excitation. Preferential selection of colour from fluorophore samples could be made using an Omega triple filter set, which gives transmission in narrow bands centred on 450 nm (blue), 520 nm (green) and 620 nm (red). The integration times were ˜30 s.

Such Ta/Al devices (resolving power ˜10-20) are capable of simultaneously measuring at least four well-separated fluorophores. Smaller band gap, lower operating temperature, STJ devices with better resolving power—e.g. Hf with R˜80 or Mo with R˜40—are potentially even better. The modest throughput of single pixel STJs can be improved by the development of large format arrays. (See Nuclear Instruments and Methods in Physics Research A 559 (2006) 782-784, “Optical fluorescence of biological samples using STJs”, G. W. Fraser, J. S. Helsop-Harrison, T. Schwarzacher, P. Verhoeve, A. Peacock and S. J. Smith).

Typically, a scanner will include an image capture/processing system, for example based upon a digital signal processor or a suitably programmed general purpose computer, and the fluorescence signals from the imaged microarray are typically output as a colour image file in an industry standard format, such as a 16 bit GIF (graphics interchange format) or TIFF (tagged image file format) format. Different fluorophores have different emission peaks and, in general, different (shorter wavelengths) absorbtion peaks. The scanner may either excite both absorption peaks simultaneously with a single wavelength, then read both emission wavelengths simultaneously, or, the microarray may be scanned at first one absorption wavelength and then at the other(s). Often lasers are employed to excite the fluorescence. It is generally preferable to read multiple fluorescence signals (colours) simultaneously as re-scanning can damage the hybridised entities and can, in particular, cause photo bleaching. Generally, passband filtering is employed to discriminate between the excitation illumination and the fluorescent emission as well as, optionally, between the different fluorescence signals.

Background material relating to microarray data analysis can be found in “Microarray data analysis: from disarray to consolidation and consensus”; David B. Allison, Xiangqin Cui, Grier P. Page and Mahyar Sabripour, NATURE REVIEWS, GENETICS, Volume 7, January 2006, page 55-65; and “Improving false discovery rate estimation”, Bioinformatics 20(11), 2004, page 1737-1745; Material published after the earliest priority date of this application can be found in “Speed-mapping quantitative trait loci using micromays”, Chao-Qiang Lai, Jeff Leips, Wei Zou, Jessica F Roberts, Kurt R Wollenberg, Laurence D Parnell, Zhao-Bang Zeng, Jose M Ordovas & Trudy F C Mackay, NATURE METHODS, Vol. 4 No. 10, October 2007, pages 841-839; and “HoughFeature, a novel method for assessing drug effects in three-color cDNA microarray experiments”, Hongya Zhao and Hong Yan, 17 Jul. 2007, BMC Bioinformatics 2007, 8:256, doi: 10.1186/1471-2105-8-256.

Conventionally, the processing of fluorescence signals from a microarray has been based upon some implicit assumptions, in particular that there is a linear relationship between the fluorescent signals from a particular spot and the relative abundances of the labelled sample or target. The inventors have, however, recognised that this is not generally true and that coupling between two different fluorophores will introduce non-linearities. The inventors have further recognised that non-linearities can occur even in a single fluorophore system. The degrees of non-linearity will in part depend upon the fluorophores employed. We will describe techniques by which these non-linearities can be taken into account. More particularly, we will describe both techniques for improved processing of signals from entities labelled or associated with two different fluorophores, and techniques relating to the determination of an optimum degree of labelling i.e. one which produces maximum brightness.

### SUMMARY OF THE INVENTION

According to a first aspect of the invention, there is therefore provided a method of determining respective first and second degree-of-labelling signals for different respective first and second fluorophores associated with a common entity, the method comprising: determining a first fluorescence signal from said first and second fluorophores under first conditions; determining a second fluorescence signal from said first and second fluorophores under second conditions different to said first conditions; and determining said first and second degree-of-labelling signals for said first and second fluorophores from said first and second fluorescence signals; and wherein said determining of said first and second degree-of-labelling signals is responsive to at least one coupling value (c₁₂; c₂₁) representing a coupling of energy between said fluorophores, or the absorption of light emitted by one fluorophore by the other fluorophore.

The skilled person will understand that in a microarray experiment signal intensities (corrected for the effects of non-linearity) are measured. The degree of labelling may be, for example, either the number of fluorophores on a particular molecule or entity or the number of fluorescent labelled molecules which bind to an entity. An example of the first case is where multiple fluorophores bind, at spatial intervals, to DNA. An example of the second case is where, say, an antibody has multiple binding sites and binds to a plurality of molecules simultaneously each carrying a single fluorophore. Embodiments of the technique can still further be used in a situation where the degree of labelling signals associated with a common entity arise from a physical mixture of different fluorophores attached to different individual molecules or entities of the same type. An example of this is where a microarray spot contains a physical mixture of the same conjugate molecule some of which have one or more fluor A moieties attached and others of which have one or more fluor B moieties attached.

Embodiments of the technique allow the degree-of-labelling signals from two different fluorophores to be separated, thus dispensing with the need for re-scanning and minimising the deleterious effects of photo bleaching. In some particularly preferred embodiments, the technique is employed with fluorescence signals from a superconducting tunnel junction detector device as described above. The techniques are particularly advantageous with this type of detector because of the relatively small number of photons which may be detected. However, more generally, embodiments of the method may be employed with any type of microarray scanning system, as well as in the context of other systems in which optical emission from spatially substantially coincident fluorophores may be observed. Thus, for example, the method may be embodied as computer programme code to implement a front end for conventional microarray scan analysis software. The degree-of-labelling signals determined for the first and second chromophores (fluorophores) may either comprise a degree-of-labelling per se, or, for example, separated signals from the two fluorophores from which respective degrees of labelling or other hybridisation information may later be derived.

In preferred embodiments of the method, at least one coupling value represents a coupling between light emitted by one of the fluorophores and absorbed by the other of the fluorophores.

In embodiments of the method the first fluorophore has an emission peak at a longer wavelength than that of the second fluorophore and the coupling value represents a coupling between light emitted by the second fluorophore and absorbed by the first fluorophore; in embodiments of the method coupling in the other direction may be substantially neglected.

The conditions under which the first and second fluorescence signals are determined generally define one or both of different illumination wavelengths and different detection wavelengths for the determination of the first and second fluorescence signals. Thus, a common illumination signal may be applied to a microarray in a single scan, using different wavelengths or wavelength bands, for example selected by filters, to determine the two fluorescence signals.

In embodiments an estimate for the one or more coupling values may be determined by performing a calibration over a range of combinations of the first and second fluorophores in different proportions. In preferred embodiments of the method the determining of the two degree-of-labelling signals also takes into account respective parameters for the two fluorophores representing a respective degree of self-quenching. Such parameters may be available or derivable from published data or may again be determined by performing a calibration, here for each fluorophore separately.

The skilled person will appreciate that the techniques we describe may be applied to a range of entities with which the first and second fluorophores are associated. For example, in a microarray experiment the two fluorophores may be associated with a common probe entity to which the separately tagged targets are attached, for example to determine a degree of relative hybridisation. Additionally or alternatively the two fluorophores may be associated with a common sample or target entity. Typically the two fluorophores will be part of a probe-target experiment, but, potentially, they may also be incorporated into the structure of a common molecule, for example as differently fluorescently tagged bases in a strand of DNA or RNA.

In some preferred embodiments the method is employed to process fluorescence data from a microarray. Generally this will comprise a microarray of DNA or RNA, although the microarray may additionally or alternatively comprise antibodies or antigens; in other applications the technique may be employed to process fluorescence data from a sandwich assay.

Thus in a further aspect there is provided a method of processing fluorescence data from a microarray, the microarray being labelled with two or more different fluorophores, the method comprising: inputting said fluorescence data, the fluorescence data representing fluorescence signals from said microarray at two or more wavelengths; determining data representing a line of parity for said fluorescence signals, said line of parity being a line along which signal intensities from fluorescence at said two or more wavelengths are expected to represent substantially equal quantities of the entities to which the fluorophores are attached; and correcting a said fluorescence signal from said microarray at one of said wavelengths using said determined line of parity.

The skilled person will appreciate that the technique may be extended to three or more fluorophores, in which case the concept of a line of parity may be extended accordingly (i.e. to a surface of equivalence, or set of such surfaces). Thus in the above method “line of parity” includes “surface of parity”. In embodiments of the method the fluorescence signal corresponds to one or more biological parameters, for example a level of gene expression or the like. In embodiments of the method is preferable that at one of the fluorescence signals used for determining the line of parity represents a control level of fluorescence (although this is not essential since one signal may be used as a control for the other even where both represent a level of a biological parameter). Where one signal is used as a control, the other generally represents a level of a biological parameter, as previously mentioned.

In embodiments the determining of the line of parity comprises determining first and second end points of the line, optionally excluding outlier data signals. One end point may correspond to fluorescence intensity signals at first and second wavelengths being substantially zero (in terms of the later equations, assuming n is approximately unity). Thus one point on the line of parity may be determined using:

S_{G}/S_{R}=(a_{G}−b_{G})/(a_{R}−b_{R})

where a and b represent characteristics of a fluorophore, the subscripts G and R representing fluorophores which fluoresce primarily at first and second respective wavelengths, for example a green fluorophore such as Cyanine 3 fluorescent dye Cy3® and a red fluorophore such as Cyanine 5 fluorescent dye Cy5®.

In preferred embodiments a second point on the line is determined using:

S_{G}/S_{R}=(b_{G}+c_{RG})/b_{R}

Where c_{RG} accounts for emission from one fluorophore which is absorbed by the other fluorophore. More particularly the c_{RG} term takes account of quenching of a short wavelength fluorescence emitter by a longer wavelength fluorescence emitter (so the longer wavelength emitter is less perturbed by this effect than the shorter wavelength emitter). In some preferred embodiments a value for c_{RG} may be determined from the fluorescence data, for example by a best-fit technique. The point on the line of parity determined by this method may be a maximum fluorescence end point, that is where the fluorescence intensity signals at the two or more wavelengths are substantially at a maximum.

In preferred embodiments the correcting process comprises compensating for a difference between a measurement variable (i.e. a non-control) fluorescence signal and a value of that fluorescence signal predicted from the line of parity, for example by subtracting one from the other. In some preferred embodiments the method also comprises correcting for systematic noise comprising one or both of: fixed pattern noise from the microarray, and noise resulting from division of one digital number by another. The fixed pattern noise may arise, for example, from artefacts due to deposition of the microarray and may have a cyclic repetition, for example at row or column sub-array intervals.

Thus, in a further aspect the invention provides a method of processing fluorescence data from a microarray, the method comprising: inputting said fluorescence data, the fluorescence data representing fluorescence signals from said microarray at a plurality of different spot locations: and processing said fluorescence data to determine a biological parameter associated with fluorescence from a said spot; and wherein said processing includes: compensating said fluorescence data for systematic noise comprising one or both of fixed pattern noise from said microarray and noise resulting from the division of one digital number by another.

The invention further provides processor control code, in particular on a carrier, to implement embodiments of the above described method. The carrier may comprise a disc such as a CD (compact disc)- or DVD (digital video disc)-Rom, programmed memory such a read only memory, or a data carrier such as an optical or electrical signal carrier. The processor control code may comprise source, object or executable code in any conventional programming language, for example C, or code for a hardware description language. As the skilled person will appreciate such code and/or associated data may be distributed between a plurality of coupled components in communication with one another.

The invention further provides apparatus configured to implement a method as described above. In general such apparatus comprises an input to receive fluorescence data to be processed, an output to provide the processed fluorescence data, either for further analysis or, for example, as a set of gene expression levels, and a data processor coupled to the input and the output, to working memory, and to program memory storing processor control code to implement a fluorescence data processing method.

In a related aspect, the invention provides apparatus for determining respective first and second degree-of-labelling signals for different respective first and second fluorophores associated with a common entity, the apparatus comprising: means for determining a first fluorescence signal from said first and second fluorophores under first conditions; means for determining a second fluorescence signal from said first and second fluorophores under second conditions different to said first conditions; means for determining said first and second degree-of-labelling signals for said first and second fluorophores from said first and second fluorescence signals; and wherein said means for determining said first and second degree-of-labelling signals is responsive to at least one coupling value (c₁₂; c₂₁) representing a coupling of energy between said fluorophores.

The inventors have further recognised that the above-described techniques may also be employed to determine an optimum degree-of-labelling of an entity with a fluorophore and, more particularly, by two or more fluorophores. This is advantageous because a procedure to label entities with one or more fluorescent tags generally involves multiple labelling experiments which are tedious and time consuming. The inventors have recognised that, depending upon the data available, only a single labelling experiment or, potentially no labelling experiments may be necessary.

Thus, according to a further aspect of the invention, there is provided a method of labelling an entity with a fluorophore, the method comprising inputting a first parameter dependent on a light creation efficiency of said fluorophore; inputting a second parameter dependent on a degree of self-quenching of said fluorophore; determining an estimate of an optimum degree-of-labelling of said entity by said fluorophore using said first and second parameters; and labelling said entity with said fluorophore in accordance with said estimated optimum degree-of-labelling.

In embodiments the estimated optimum degree-of-labelling comprises an estimated degree-of-labelling (number of fluorophores per entity, e.g. molecule) at which fluorescence intensity (brightness) is predicted to a maximum.

Preferably the first parameter is further dependent on one or more of a structure of the entity, a structure of the fluorophore, and a bonding between the fluorophore and the entity. In some particularly preferred embodiments, the method is employed with a plurality of fluorophores, using a third parameter dependent upon the degree of coupling between at least two of the plurality of fluorophores to determine an estimated optimum degree-of-labelling for each of the fluorophores in the presence of the other. The degree of coupling may be estimated, for example, by performing a calibration experiment using entities labelled with a range of different respective combinations of the plurality of fluorophores.

In a further aspect the invention provides an entity labelled with one or more fluorophores using the above described method. Thus in embodiments the entity has a substantially optimum degree-of-labelling by the one or more fluorophores. The optimum degree-of-labelling may be defined as a degree-of-labelling which corresponds to substantially the maximum fluorescent light yield from the labelled entity for the relevant fluorophore.

Thus in further aspects the invention provides an entity labelled with a plurality of different fluorophores, and a kit of fluorescent probes, respective numbers of said different fluorophores being such that a fluorescence signal (S(n)) from each said fluorophore is substantially maximised.

The invention still further provides a fluorophore-labelled entity having a number of labelling fluorophores determined by a product of a figure of merit (R), an example of which is described later, multiplied by a constant of proportionality determined from a measured set of peak values of fluorescence against respective figures of merit for a plurality of different other fluorophores. For example the constant of proportionality may be that shown in FIG. 7, discussed later, +/−50%.

The invention still further provides a method of manufacturing a kit of fluorophore labelled probes, the method comprising: determining a combination of fluorophores for said kit; and manufacturing said kit using said determined combination of fluorophores; and wherein said determining of said combination of fluorophores comprises: selecting one or both of a set of fluorophores for said kit of fluorophore labelled probes and a degree of labelling of said probes by said fluorophores using a fluorescence brightness figure of merit function (R) for a candidate said fluorophore of the set.

In embodiments, the fluorescence brightness figure of merit function is dependent on a degree of overlap between emission and absorption spectra of a said candidate said fluorophore. Preferably R also comprises a function dependent on the quantum yield of a said candidate fluorophore, a maximum value of an extinction coefficient of the fluorophore. Additionally or alternatively said function comprises a function of i) a parameter dependent on a light creation efficiency of fluorophore, and ii) a parameter depending on a degree of self-quenching of the fluorophore, said selecting further being dependent on iii) a parameter dependent on a degree of coupling between the fluorophore and another fluorophore.

In embodiments the kit of fluorophore labelled probes comprises a calibration kit for a microarray or another diagnostic platform. In embodiments the number of calibration fluorophores matches the number of experimental fluorophores. In some preferred embodiments the kit of fluorophore labelled probes for use with an STJ detector.

The invention also provides a kit of fluorophore labelled probes comprising a kit of fluorophores, and wherein one or both of a set of fluorophores for said kit of fluorophore labelled probes and a degree-of-labelling of said probes by said fluorophores are selected using a fluorescence figure of merit function (R) for a candidate said fluorophore of the set, and wherein said fluorescence brightness figure of merit function is dependent on a degree of overlap between emission and absorption spectra of a said candidate said fluorophore.

In embodiments the fluorophores and/or degree-of-labelling of the fluorophores is selected to optimise subsequent signal detection and/or measurement and/or spectral deconvolution.

### BRIEF DESCRIPTION OF DRAWINGS

These and other aspects of the invention will now be further described, by way of example only, with reference to the accompanying figures in which:

FIG. 1 shows a comparison of measured brightness data for Alexa (Registered Trade Mark, RTM, of Invitrogen Corp.) 488 (individual squares), Fluorescein-EX (circles) and Alexa® 546 (triangles), with calculated curves based on eq. 3b and the best-fit values for the parameters a and b given in Table 2, (GAM IgG Goat Anti-Mouse (secondary antibody conjugate) based on the G structural form of immunoglobulin for the Alexa® fluorophores, streptavidin for F-EX);

FIG. 2 shows a comparison of measured and calculated brightness data for Alexa® 350 (diamonds; streptavidin conjugate), Alexa® 555 (squares; GAR Table 1 conjugate (Goat Anti-Rabbit (secondary antibody conjugate)-, AMCA (fluorophore amino-methylcoumarin acetic acid) (crosses; streptavidin) and Cy3 (open circles; streptavidin conjugate: filled circles GAR conjugate;

FIG. 3 shows a comparison of measured and calculated brightness data for Oregon Green 514 (diamonds), Oregon Green 488 (squares), FITC (fluorophore fluorescein isothiocyanate) (circles) and Rhodamine Red-X (circles), (GAM conjugate in all cases);

FIG. 4 shows a comparison of measured (individual symbols) and calculated (full curves) brightness functions for Alexa® 647-labelled conjugates: crosses—streptavidin conjugate; squares—transferrin Transferrin (a protein) circles—concanavidin A; diamonds—GAR IgG; diagonal crosses—GAM IgG; the single theoretical curve is calculated for a=1.5, b=0.14;

FIG. 5 shows a comparison of measured (individual symbols) and calculated (full curves) brightness functions for Texas Red-X-labelled conjugates

FIG. 6 shows a comparison of measured and calculated brightness data for Alexa® 532 and Rhodamine 6G, both conjugated to GAM;

FIG. 7 shows a linear relationship between measured nₚₑₐₖ and figure-of-merit R for GAM conjugates of Table 2; this figure allows the maximum of the brightness function S(n) to be estimated for any (GAM-conjugated) fluorophore for which a value of R can be constructed;

FIG. 8 shows apparatus implementing an embodiment of a technique according to the invention;

FIGS. 9a to 9c show, respectively, a microarray data scatter plot showing construction of a line of parity according to an embodiment of the invention, the plot of FIG. 9a with fluorescence data corrected for inter-fluorophore fluorescence quenching based on fluorescence level prediction from the line of parity of FIG. 9a, and a so-called MA plot (scatter plot with transformed axes) for the corrected data, also illustrating the imposition of a signal-to-noise (S/N) threshold; and

FIGS. 10a to 10d show, respectively, background noise from the HFF-PDS (Human foreskin fibroblasts, infected with the PDS strain of the parasite Toxoplasma gondii (a close relation of the organism which causes malaria)) data set folded modulo-28 illustrating artefacts due to fixed pattern noise, systematic noise arising from the division of one small digital number by another, results of a simulation illustrating how noise could be misinterpreted as gene expression, and a similar example showing real, raw microarray (from SMD data set 3932).

### DETAILED DESCRIPTION OF PREFERRED EMBODIMENTS

Broadly speaking we will describe a model of the self-quenching of fluorescent emission and compare this with measurements of light yield versus degree-of-labelling for a number of fluorophores (dyes) commonly used in biology. The model is physically based on the emission and absorption of light by molecules of the same species. The model shows that the optimum degree-of-labelling corresponding to maximum light yield, is predictable from a combination of basic parameters of the fluorophore. However the maximum can also depend on the fluorophore's conjugate molecule. Extension of the model to multi-fluorophore systems is described, as is a method for determining degree-of-labelling signals in such systems, and procedures for the recovery of biological information in such systems in the presence of non-linearities.

In dye-labelled biological systems, the fluorescent signal Iₛ is not always linearly related to the degree-of-labelling n—the number of fluorophores present per conjugate molecule. (In what follows,

$${I}_{s} = {\int }_{{λ}_{1}}^{{λ}_{2}} i ( λ ) d λ$$

formally denotes the integral of the wavelength-dependent emission function i over an output filter bandpass [λ₁≦λ≦λ₂]). The conjugate molecule is the biologically active protein or antibody to which the fluorophore is attached. The phenomenon of self quenching—the decrease in fluorescent signal observed for high labelling densities—is poorly understood [S. Hamann, J. F. Kiilgaard, T. Litman, F. J. Alavrez-Leefmans, B. R. Winther and Zeuthen, J. Fluorescence 12 (2002) 139], even though it leads in many cases to a well-defined degree of labelling (n=nₚₑₐₖ) for maximum brightness.

Of possible quenching mechanisms dynamic fluorescence quenching cannot be considered a universal process because it depends on collisional energy exchange with a quenching agent distinct from the fluorophore itself. Static quenching, by contrast, depends on the formation of non-emitting molecular complexes by the fluorophore and the quencher. The process of fluorescent resonant energy transfer (FRET), thirdly, requires the overlap of the donor emission spectrum and the absorption spectrum of the acceptor in a system whose two components are separated by only ˜nanometre distances.

Here, we assume that self quenching—leading to signal non-linearity—is primarily due to the absorption by the fluorophore molecules of their own emitted light. In other words, we suppose that, fundamentally, self-quenching arises from the overlap of the absorption and emission spectra in the same dye molecule in an otherwise transparent system. In what follows, we compare the predictions of our model with measurements of the brightness function S(n) reported in the literature, calculate nₚₑₐₖ, the labelling density which corresponds to maximum signal.

Table 1 summarises the acronyms used below to denote specific fluorophores, conjugate molecules and reference standards for the estimation of quantum yield [R. F. Rubin and A. N. Fletcher, J. Luminescence 27 (1982) 445].

TABLE 1
Acronyms of biological entities.

| | |
|---|---|
| AMCA | Amino-methylcoumarin acetic acid (fluorophore) |
| CTMR | Carboxytetramethylrhodamine (reference standard) |
| DDAO | Dodecyldimethylamine Oxide (reference standard) |
| FITC | Fluorescein Isothiocyanate (fluorophore) |
| GAM IgG | Goat Anti-Mouse (secondary antibody conjugate) |
| | based on the G structural form of immunoglobulin |
| GAR IgG | Goat Anti-Rabbit (secondary antibody conjugate) |
| S101 | Sulforhodamine 101 (reference standard) |

### Calculation of Fluorescent Intensity

Consider the absorption of light from a monochromatic source of intensity I₀ (photons/cm²/s) and wavelength λₛ in a dye-labelled biological sample whose thickness is d and whose volume is V. In a weakly absorbing system, the absorbance A due to the fluorophore may be written as either:

A=ε·d·C -(1a)

or

A=N·σ·d -(1b)

where:
ε is the extinction coefficient, in units of cm⁻¹ Mol⁻¹
C is the concentration, in Mol
σ is the wavelength-dependent absorption cross-section, in units of cm².
N is the number of fluorophore molecules per unit volume
   while, if dΩ is the solid angle which an ideal detector subtends at the sample and Q is the absolute quantum efficiency of the fluorophore, the signal intensity Iₛ (detected photons/s) is:

Iₛ=I₀AQ[V/d][dΩ/4π] -(1c)

Eqs. (1a, b) express, for biologists and physicists respectively, the same underlying Beer-Lambert law. We see from eq. (1b) that it is the number of fluorophores per unit volume, N, which quantitatively determines the degree of absorption, but note from above that the parameter almost always reported in biology is n, the number of fluorophores per conjugate molecule, measured in Mol/Mol. The relationship between N and n, however, is simple and linear if M, the mass of the conjugate molecule (e.g. 52,800 Da for the protein streptavidin) is much greater than that of the fluorophore (e.g. 300-900 Da for the fluorophores below). With Min grams:

N=[n·Nₐ·ρ]/M -(2)

where:
Nₐ is Avogadro's number and
ρ is the effective density of the conjugate molecule

Substituting for A (from eq. 1b) and N (from eq. 2) in eq. (1c), we find, after some manipulation:

Iₛ=I₀nQN_{c}σ[dΩ/4π]=I₀S(n)N_{c}σ[dΩ/4π] (3)

where N_{c} is the number of conjugate molecules in the sample and the physical brightness function S(n)=nQ incorporates the possibility that the quantum efficiency depends on the degree of labelling, n.

This analysis suggests that the brightness function S(n) should be higher, for a given n value, the lower the mass of the conjugate molecule, since then there will be proportionately more fluorophores per unit volume, provided the density varies little between conjugates. This hypothesis is tested later.

### Brightness Function

In order to estimate the form of S(n), we need to consider first the absorption of the source flux and, second, the reabsorption of the fluorescent emission by the same population of dye molecules. Applying eqs. (1b, 2), the probability of fluorescent light emission and the probability of reabsorption of that fluorescent photon within the sample are both proportional to n, the number of fluorophores per conjugate molecule. We write λₚ(>λₛ) for the wavelength corresponding to the peak of the fluorophore's emission spectrum.

Thus, disregarding details of both the sample geometry and of the interaction of the fluorophore with its host molecule, the brightness function, in arbitrary units, is then found from the product of the production and reabsorption probabilities as follows:

S=[k₁n][1−k₂n] -(4a)

which is of the form:

S=an−bn² -(4b)

where: a=k₁ and b=ak₂ are characteristics of the fluorophore. A close identification of the former constant follows from the biological definition of brightness function:

S(n)=n·RQY(n) -(5)

where RQY(n) denotes the relative quantum yield for a degree-of-labelling, n. From eq. (4b). it follows that:

Lim_{n→0}(S(n)/n)=Lim_{n→0}RQY(n)=a -(6)

is Differentiating eq. (4b) we find that, if nₚₑₐₖ is the value of n which corresponds to the maximum light yield, we have:

$${n}_{peak} = \frac{1}{2 {k}_{2}} = \frac{a}{2 b} \qquad ( 7 )$$

The optimum degree-of-labelling can therefore be estimated for any fluorophore for which the constants a and b (or simply k₂) have been determined. The same analysis gives the maximum useful labelling density, n_{zero}, for which the signal is totally quenched:

n_{zero}=2nₚₑₐₖ -(8)

Thus, a defining characteristic of a fluorophore exhibiting self quenching by self-absorption is that the maximum degree of labelling is exactly twice the value corresponding to maximum light yield. Formally, this result is in conflict with the starting mathematical assumption of weak absorption and with the physical observation that, for most fluorophores, the absorption spectrum does not completely overlap the emission spectrum. The physical (above) and biological definitions of brightness function differ only by a multiplicative factor which is the absolute quantum yield of a reference standard (see below).

Comparison with Published Data

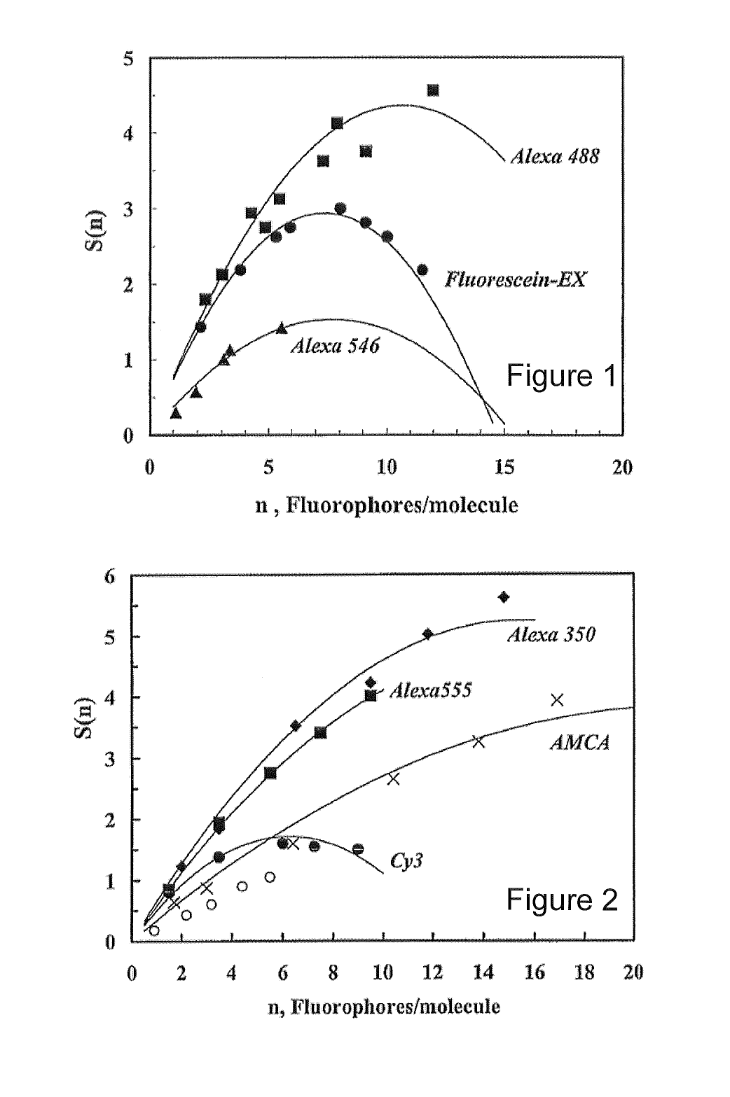

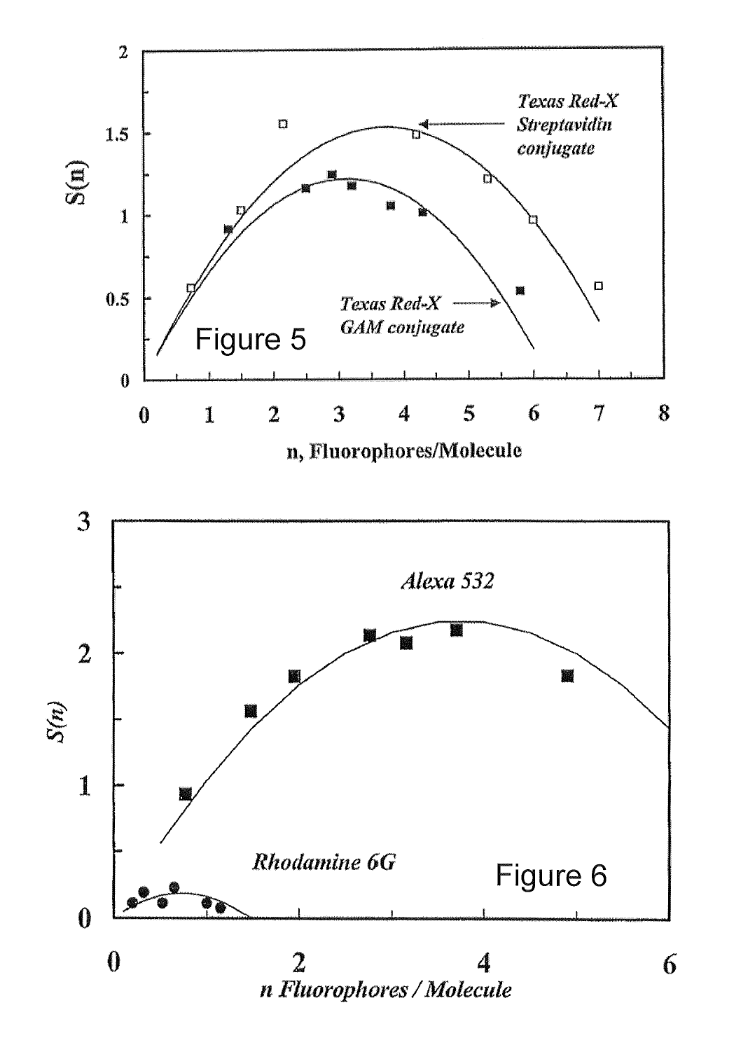

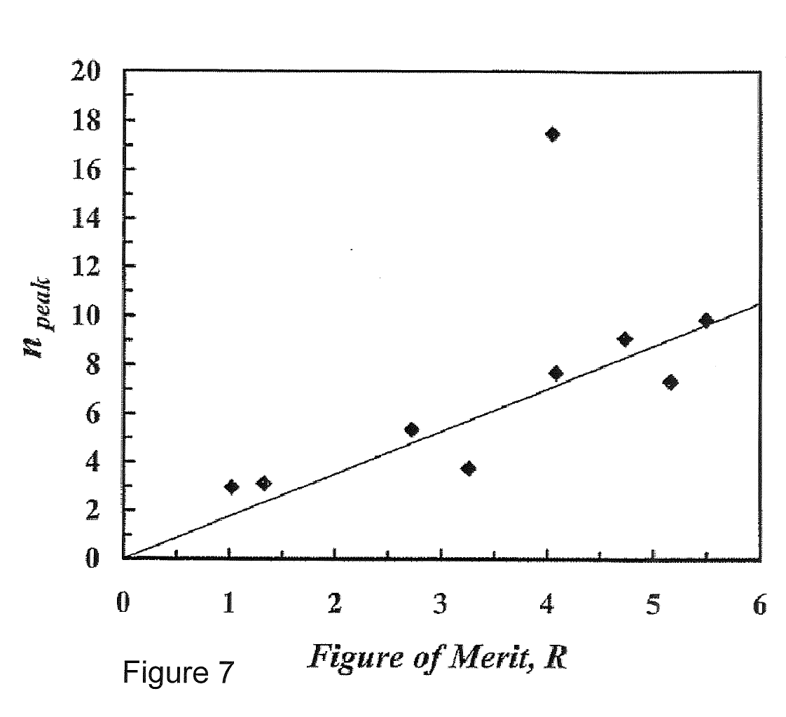

FIGS. 1 to 6 compare published S(n) data sets with calculations based on eqn. (4b). We see that, for determined values of a and b, the universal function derived from a “self-absorption” model of self quenching is well supported by measurements on a number of well-known dyes, conjugated to a variety of biomolecules [B. Randolph and A. S. Waggoner, Nucleic Acids Research 25 (1997) 2923; N. Panchuk-Voloshina and seven co-authors, J. Histochemistry and Cytochemistry 47 (1999) 1179; H. J. Gruber and seven co-authors, Bioconjugate Chem. 11 (2000) 696; J. E. Berlier and fourteen co-authors, J. Histochemistry and Cytochemistry 51 (2003) 1699; Invitrogen (formerly Molecular Probes Inc., Oregon) Online Handbook, Section 1 http://www.probes.com/handbook/sections/0001.html; C. Lefevre, H. C. Kang, R. P. Haugland, N. Malekzadeh, S. Arttamangkul and R. P. Haugland, Bioconjugate Chem. 7 (1996) 482]. The F-EX and Texas Red-X data sets (FIGS. 1 and 5 respectively), in particular exhibit maximum light yields at half the degree of labelling corresponding to total quenching.

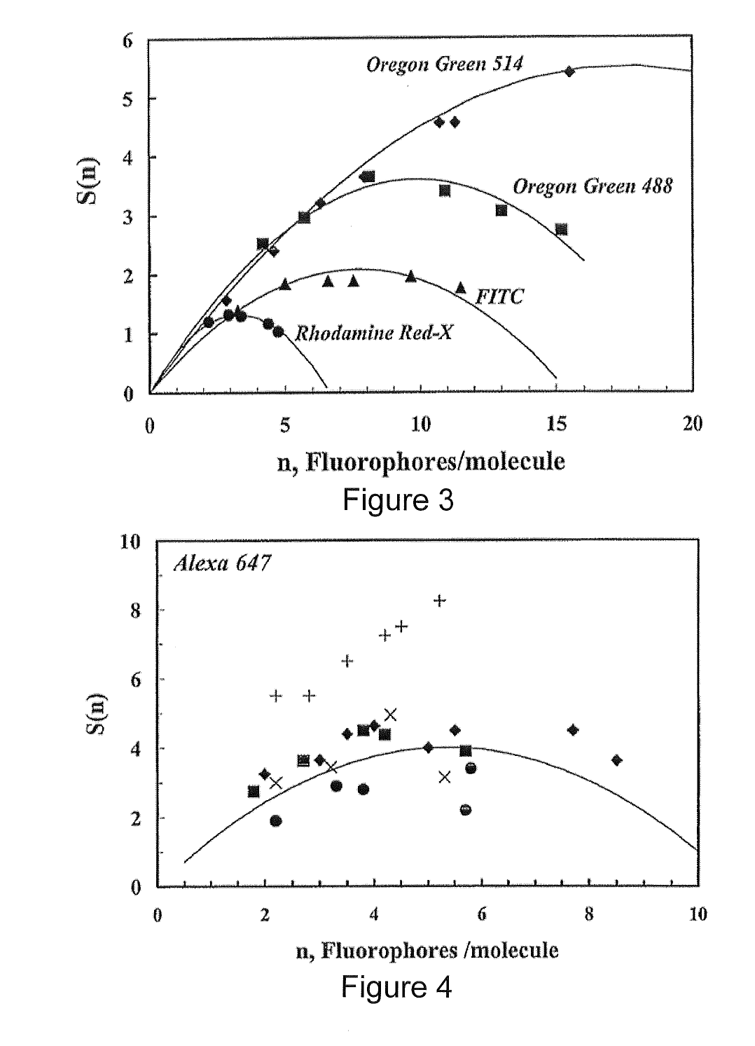

FIGS. 4 and 5 also indicate that maximum light yield occurs at higher n values for fluorophores conjugated to the protein streptavidin than for (much heavier) secondary antibody conjugates such as GAM (defined in Table 1). The masses of these antibodies are difficult to find in the literature, but a single heavy (H) chain of immunoglobin (Ig) with a mass of ˜50,000 Da alone weighs approximately the same as streptavidin. Thus, our prediction that the fluorescent signal should depend inversely on the mass of the conjugate molecule, appears confirmed. The other protein represented in FIG. 3—Concanavidin A—is about twice as heavy at 104 kDa as streptavidin.

### Prediction of the Self-Quenching Properties of New Fluorophores

It is useful to be able to predict the optimum degree of labelling for new fluorophores, given only basic physical data.

Returning to eq. (4), the fluorophore constant k₁ accounts for the details of the fluorescent light creation process and for the wavelength-dependent losses of fluorescent light in a given absorber geometry; it includes the subtleties of the chemical bonding between the fluorophore and its conjugate biomolecule. In terms of measurable quantities, therefore, k₁ is, as already demonstrated (eq. (6)), related to the quantum yield Q (fluorescent photons/absorbed photon), and to the maximum value of the extinction coefficient, εₘₐₓ. The fluorophore constant k₂, by contrast, should also account for the degree of overlap between the emission and absorption spectra of the fluorophore. One measure of this overlap is the value of the extinction coefficient at the wavelength of maximum emission, ε(λₚ). One may argue, therefore, that the combination of measurable quantities:

R=Qεₘₐₓ/ε(λₚ) -(9)

should track nₚₑₐₖ, if the self-absorption model of self-quenching is correct.

In order to test this hypothesis, we use values of the absolute quantum yield Q, measured (ideally) for a common degree-of-labelling, n=1. Readily available efficiency values usually refer to the pairwise comparison of spectrally similar fluorophores and are measured relative to a reference standard (such as S101) for degrees-of-labelling chosen to match the absorbances of the fluorophore pair. Table 2 (below) gives the values for relative quantum yield RQY(n) and the correction factors used to arrive at the desired absolute quantum yield Q(n=1) via:

TABLE 2
Derived model parameters for fluorophores conjugated to GAM IgG or
streptavidin (denoted G or S in the leftmost Column).

| | | | | | | | | | | | | −(10) |
|---|---|---|---|---|---|---|---|---|---|---|---|---|
| $Q ( n = 1 ) = [ RQY ( n ) \cdot {Q}_{ref} ] [ \frac{1 - b / a}{1 - n ( b / a )} ]$ | | | | | | | | | | | | |
| | | | | | | | | | | | Extinction | |
| Fluorophore/ | | | | | | Standard | | Scale | Qrel | Qabs | Coefficent | |
| Conjugate | a | b | a/2b | RQY(n*) | n | fluorophore | Qref | Factor | (n = 1) | (n = 1) | Ratio | R |
| Alexa 488 (G)[3] | 0.82 | 0.045 | 9.11 | 0.6 | 5.3 | Fluorescein | 0.97 | 1.33 | 0.8 | 0.776 | 3.6 | 2.79 |
| Alexa 532 (G) | 1.75 | 0.255 | 3.43 | 0.59 | 3.7 | Rhodamine | | | | 1.039 | 3.3 | 3.43 |
| Alexa 546 (S) | 0.4 | 0.026 | 7.69 | 1.25 | 4 | CTMR | 0.68 | 1.26 | 1.579 | 1.074 | 2.08 | 2.23 |
| Alexa 594 (G) | | | 5.0 | 0.53 | 4 | S101 | 0.9 | | | | 5 | 0 |
| Oregon Green 488 | 0.73 | 0.037 | 9.86 | 0.43 | 5.5 | Fluorescein | 0.97 | 1.32 | 0.566 | 0.549 | 10 | 5.49 |
| (G) | | | | | | | | | | | | |
| Texas Red-X (G) | 0.78 | 0.125 | 3.12 | 0.27 | 3.9 | S101 | 0.9 | 2.24 | 0.605 | 0.544 | 2.44 | 1.33 |
| FITC (S) | 0.54 | 0.035 | 7.71 | 0.23 | 4.4 | | 0.97 | 1.31 | 0.30 | 0.29 | 8.33 | 2.43 |
| Rhodamine Red-X | 0.8 | 0.121 | 3.31 | 0.16 | 4.6 | S101 | 0.9 | 2.79 | 0.45 | 0.40 | 2.54 | 1.02 |
| (G) | | | | | | | | | | | | |
| Fluorescein EX | 0.8 | 0.054 | 7.35 | 0.23 | 4.4 | Fluorescein | 0.97 | 1.33 | 0.31 | 0.297 | 8.33 | 2.47 |
| (G)[3] | | | | | | | | | | | | |
| Alexa 568 (G) | | | | 0.37 | 4.2 | S101 | 0.9 | | | 0 | 3.85 | 0 |
| Oregon Green 514 | 0.63 | 0.018 | 17.5 | 1 | 1 | 0 | 1 | 1 | 1 | 1 | 5.5 | 5.5 |
| (G) | | | | | | | | | | | | |
| Rhodamine 6G (G) | 0.5 | 0.34 | 0.74 | 0.03 | 3 | R6G | 0.95 | 1 | 0.03 | 0.0285 | 9.23 | 0.26 |
| Alexa 647 (G) | 1.5 | 0.14 | 5.36 | 1 | 1 | DDAO | 1 | | 1 | 1 | 2 | 2 |

Thus, we are able to construct, for eight fluorophores conjugated to GAM for which all the basic data is available, the relationship between nₚₑₐₖ and the figure of merit R shown in FIG. 6. Apart from one outlier, the relationship is linear, enabling us to predict values for nₚₑₐₖ for two further fluorophores—Alexa® 568 and Alexa® 594—for which no estimate of R is available. For Alexa® 568, R=1.28, implying that the brightness function should have a maximum at ˜2 fluorophores/molecule. For Alexa® 594, R=2.39, implying a peak at n ˜4.

### Extension to Two or More Fluorophores

If two fluorophores (subscripted 1 and 2) label the same conjugate molecule to degrees n₁ and n₂ respectively, and both fluorophores independently exhibit self-quenching due to self-absorption, the brightness function S will have the form:

S(n₁,n₂)=a₁n₁−b₁n₁²+a₂n₂−b₂n₂²−(c₁₂+c₂₁)n₁n₂ -(11a)

The term c₁₂ accounts for emission from fluorophore 1 being absorbed by fluorophore 2 while c₂₁ describes emission from fluorophore 2 being absorbed by fluorophore 1. In other words, there is likely to be mutual quenching observed in the two signal channels.

Typically, the fluorophores chosen for a dual labelling experiment have very distinct (absorption and) emission spectra. Measuring in a well-defined bandpass, one would therefore hope to see the signature of only one fluorophore of the pair, but our model indicates otherwise. In the bandpass appropriate to fluorophore number 1:

S₁(n₁,n₂)=S(n₁)−c₁₂n₁n₂<S(n₁) -(11b)

and

(Iₛ)₁=I₀S₁(n₁,n₂)N_{c}σ₁[dΩ/4π] -(11c)

with similar expressions for fluorophore number 2:

S₂(n₁,n₂)=S(n₂)−c₂₁n₁n₂<S(n₂) -(11d)

(Iₛ)₂=I₀S₂(n₁,n₂)N_{c}σ₂[dΩ/4π] -(11e)

A number of points emerge from this discussion, which have useful implications for the interpretation of (for example) DNA microarray data.

Even in the complete absence of spillover of the emission spectra between output bandpasses, dual (or, by extension, multiple) labelling experiments can be expected to exhibit inter-dependent signal intensities in the output channels because of mutual quenching. The fluorescent intensity from one fluorophore, its concentration held constant, is reduced by an increase in the concentration of a second fluorophore.

The signal intensities described in eqs. (11c) and (11e) therefore lead to underestimates of the “true” degrees of expression in a microarray experiment. Furthermore, if the fluorophores are ordered by increasing wavelength of maximum emission, then we expect that c₂₁<<c₁₂. In other words, the response of the red fluorophore in a dual labelling experiment should be less perturbed by the presence of the second species than that of the blue fluorophore.

The skilled person will also understand that the above considerations can be used to select two or more fluorophores for a kit of fluorophores for detector calibration. The fluorophores may, for example, be selected to optimise (maximum) the signal from each according to the above equations, optimally taking into account detector sensitivity.

These predictions can be subject to experimental test in the form of a dilution experiment, in particular with an STJ detector, use of a superconducting tunnel junction (STJ) detector permitting the registration of fluorescent spectra on a photon-by-photon basis from DNA labelled, in proportions ranging from 1:4 to 4:1, with both Alexa® 488 (emission in the 510-543 nm band of a triple band output filter) and Cy3 (emission in a longer wavelength band 607-659 nm). The counting rates in these two bands are summarised in Table 3 (below). The ratio of count rates follows the dilution ratio more or less linearly, although the dynamic range is only 6.4:1, rather than the 16:1 expected from the known amounts of fluorophore. Of more interest in the present context is the decrease (from 0.45 to 0.27 to 0.32 counts/second) in the Alexa® 488 signal as the amount of Cy3 present is increased above parity and the decrease in the Cy3 signal (from 1.85 to 0.8 to 1.39 counts/second) as the amount of Alexa® 488 increases. These results could be artefacts of subtle variations in the absolute amounts of dye present in the various samples, but the suppression of one fluorophore's signal intensity by the increased presence of a second fluorophore is also clearly embodied in eqs. (11a-e).

These results suggest that the full recovery of biological information from multiple fluorophore systems (such as microarrays) will benefit from preparatory calibration experiments of the kind summarised in Table 3, in order to account for non-linear behaviour due to self- and mutual quenching.

TABLE 3

| | Alexa488/ | | | | |
|---|---|---|---|---|---|
| | Cy3 Ratio | | | | |
| | 4:1 | 2:1 | 1:1 | 1:2 | 1:4 |
| Count rate in | 0.88 | 0.27 | 0.45 | 0.27 | 0.32 |
| short wavelength | | | | | |
| band (s⁻¹) | | | | | |
| Count rate in long | 1.39 | 0.80 | 1.85 | 1.47 | 3.14 |
| wavelength | | | | | |
| band (s⁻¹) | | | | | |
| Ratio | 0.64 | 0.34 | 0.245 | 0.18 | 0.10 |

The skilled person will appreciate that the brightness function described above may be employed in a number of different methods. For example a value for c₁₂ (and optionally also c₂₁) may be determined by a calibration procedure in which the combined brightness function S is measured for a range of different values of n₁ and n₂. Then a value for S(n₁,n₂) may be measured and, knowing c₁₂, a value for S(n₁) may be determined; and similarly for S(n₂). Using equation 11a self-quenching may be taken into account (through b, which is related to k₂).

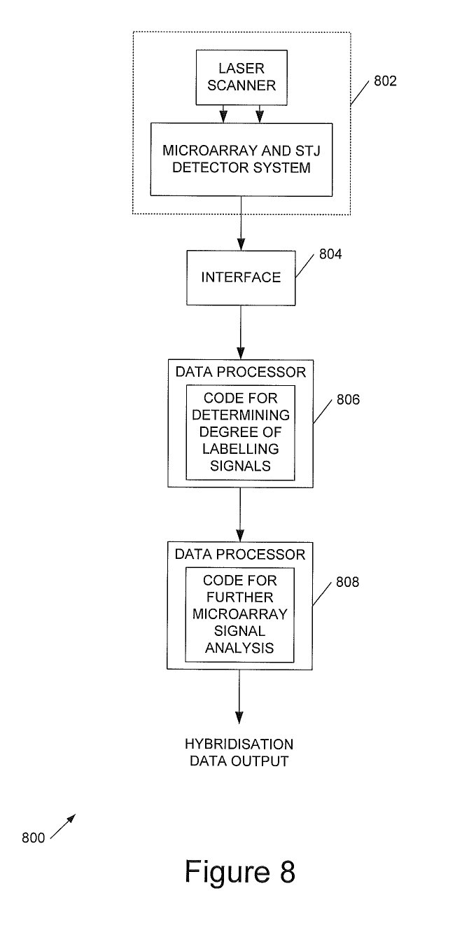

FIG. 8 shows a block diagram of a microarray scanner and analysis system 800 configured to implement an embodiment of the described method. The system includes a microarray scanner 802 employing a superconducting tunnel junction detector, coupled to an interface 804 which provides an output to a data processor 806 storing code for determining degree-of-labelling signals for an entity tagged with two different fluorophores, in accordance with equation with 9b. The output of this data processor is provided to a further data processor 808 to further analyse the fluorescence data from the microarray.

### Application to Microarray Analysis

The desired result of an example two-colour microarray analysis is the identification of those genes which are significantly over- or under-expressed in a disease or experimental state relative to a normal or control state. The experimental state signal is represented by the fluorescent intensity in one colour channel (Green, represented by the fluorophore Cy3, in the example below) relative to the intensity in a second colour channel (Red, represented by Cy5). The crucial step in any microarray analysis, therefore, is to establish a line of parity—the locus of points where degrees of expression in the two channels—represented externally by the signal intensities—are equal. The above analysis suggests an unambiguous method to closely approximate the line of parity.

Let the ratio of experimental to control intensities be denoted G/R. Then:

G/R≈S_{G}(n)/S_{R}(n)=[a_{G}n−(b_{G}+c_{RG})n²]/[a_{R}n−b_{R}n²] -(12)

This general expression is not particularly useful, since it is not easy to identify physical values of n in a microarray context, but one can identify two limiting cases which are useful—as n tends to unity, when:

G/R≈[a_{G}−b_{G}−c_{RG}]/[a_{R}−b_{R}] -(13a)

and as n tends to infinity, when:

G/R≈[b_{G}+c_{RG}]/[b_{R}] -(13b)

For any given fluorophore there are two wavelength-independent constants: a and b. In the above equations the subscript G denotes the green fluorophore (e.g. Cy3). The subscript R denotes the red fluorophore (e.g. Cy5). The values for a_{G}, b_{G}, a_{R} and b_{R} are determined independently from analysis of published curves of light yield versus degree-of-labelling. The remaining coupling constant (c_{RG}) expresses the mutual self-quenching between fluorophores and is a free parameter fixed by fitting to the data.

All the parameters in eqs. (13a, b) are known from the earlier characterisation of the individual Cy3 and Cy5 fluorophores, except c_{RG}, which can safely be approximated by zero in the dilute case, eq. (13a), but not in the intensive case, eq. (13b). Thus, we have limiting expressions for the local line of parity, with only one free parameter to be fit to data (c_{RG}), and with no a priori assumptions regarding the biology of the system.

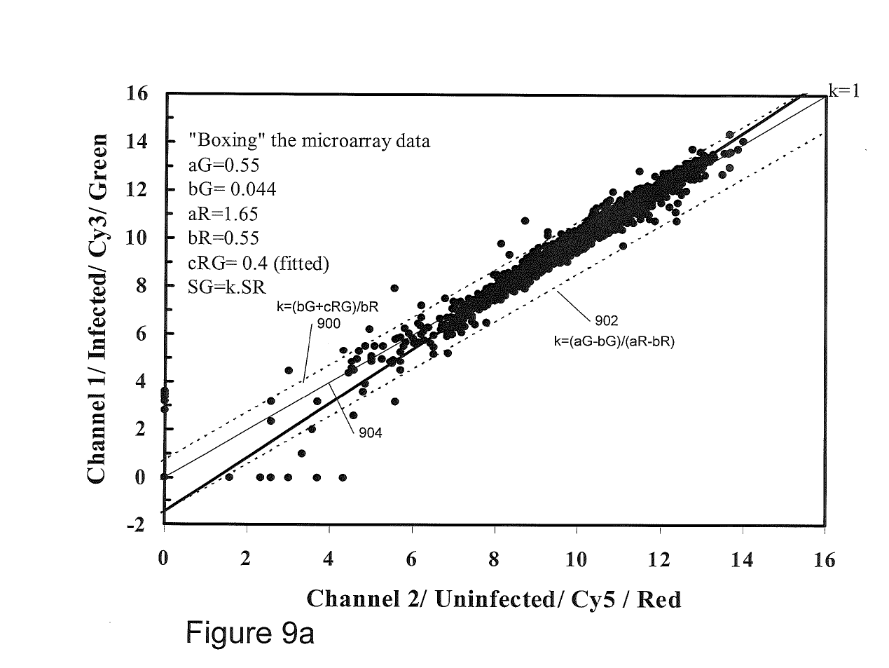

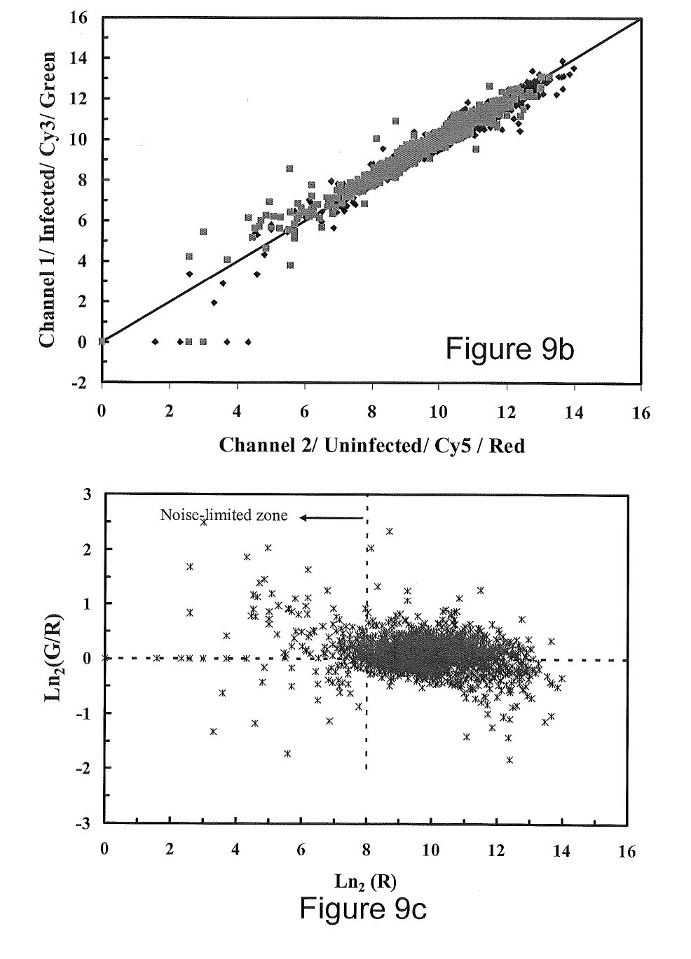

The procedure is illustrated in FIGS. 9(a,b) using a dataset from the Stamford Microarray Database (SMD). The first 1000 genes (ex 23,000 total) from the dataset SMD 3932 are plotted in a (base 2) log-log space and the upper 900 and lower 902 “tangent” lines found from eqs. 13a, b with the values of the fluorophore parameters recorded in the inset and shown below:

a_{G}=0.55

b_{G}=0.044

a_{R}=1.65

b_{R}=0.55

c_{RG}=0.4 (fitted)

S_{G}=K·S_{R}

For line 900, k=(b_{G}+c_{RG})/b_{R}

For line 902, k=(a_{G}−b_{G})/(a_{R}−b_{R})

For line 904, k=1

The overall line of parity is taken to be the diagonal straight line joining the bottom left (bottom of line 902) and top right (top of line 904) of the “box” containing the bulk of the data points.

FIG. 9(b) shows the results of collapsing the data onto this line of parity, by subtracting on a point-by-point basis the difference between the measured Green signal intensity and that predicted from the Red signal intensity and a knowledge of the true line of parity. FIG. 9(b) also shows that the best-fit to the first 1000 data points is good also for the next 1000 in the sequence (the different shaped data points represent two different 1000—point data sets).

Thus the above example uses the limiting cases of the fluorophore brightness function ratio S(n)Cy3/S(n)Cy5 to “box” the microarray data scatter plot. Joining the corners of the box gives a good approximation to the true line of parity, taking account of the non-linearity of the two fluorophore responses. Then the data is collapsed onto the nominal line of parity by calculating the difference in the y-axis between the data point and the line of parity established. FIG. 9c shows an MA plot of the ln 2 ratio versus the control Cy5 signal, (not versus the average of Cy3 and Cy5, because if there is any gene expression averaging will make the x-axis noisier), after the transformation, with the identification of the unity signal-to-noise ratio S/N line (imposition of a unity S/N threshold). Optionally there may then be rejection of points based on systematic, for example, fixed pattern noise.

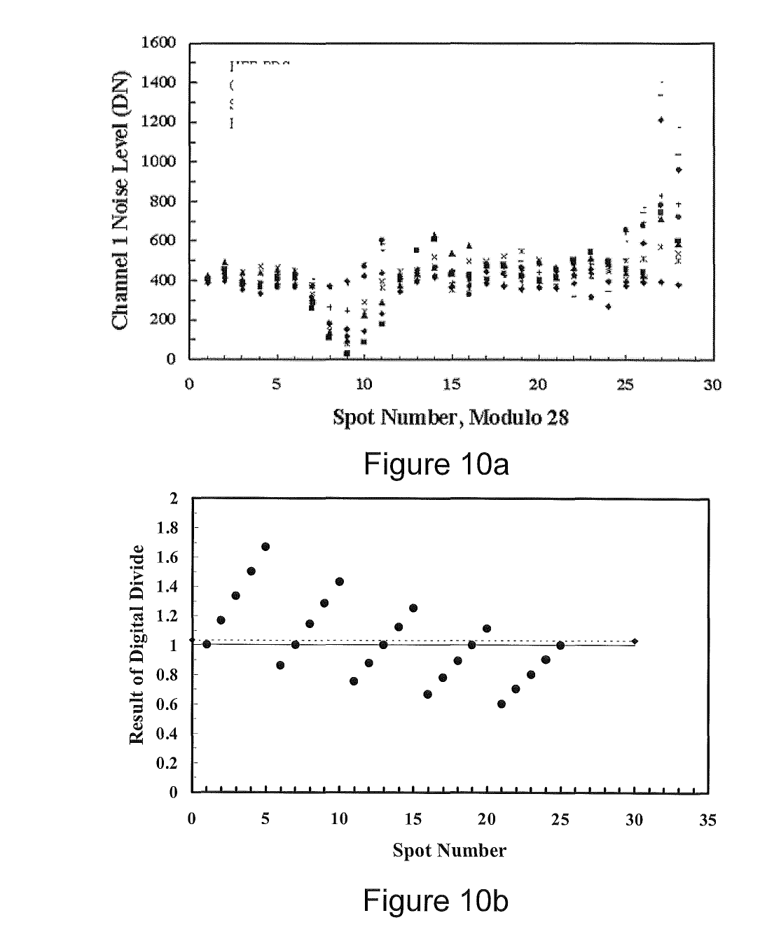

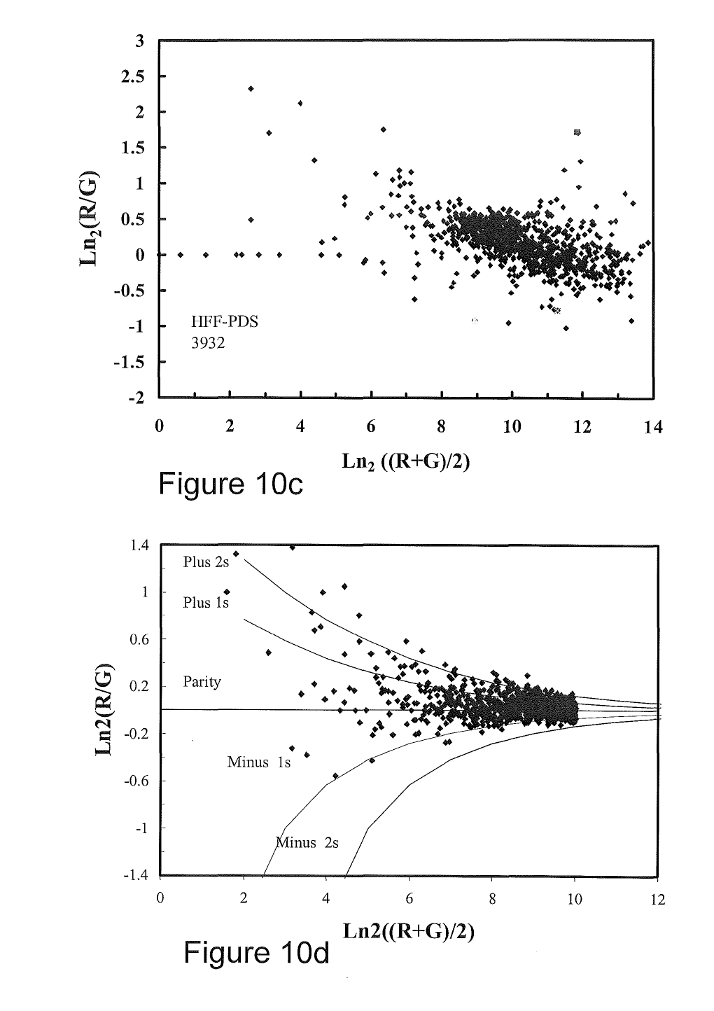

There is evidence for fixed pattern noise related to the 28×27 sub-array pattern of the microarray data for the system HFF-PDS (no. 3932). In FIG. 10a, folding the data series modulo (in this example) 28 produces a cyclic deviation of the noise in signal channel 1 from its mean. Thus channels 8-10 and 25-28 in every cycle may be flagged as unreliable. There is also evidence at low signal levels for “preferred locations” associated with the division of one digital number by another. FIG. 10b shows the 25 possible outcomes of dividing two integers in the range (8±2) one by the other. Further, the spread in signal levels at the low end is explicable by assuming exact parity (R=G) perturbed by Poissonian statistics in both channels. This is confirmed by the simulation shown in FIG. 10c, although this produces what might be identified as “over” and “under” expressed genes.

FIG. 10c provides a model template to which the real data can be transformed. FIG. 10d shows the raw 3932 data set overlaid on this template.

An analysis therefore preferably includes a systematic error analysis and correction, in particular one or more of: removal of data points corresponding to fixed pattern noise locations, in particular Modulo x elimination of data points corresponding to fixed pattern noise locations (in the example above, x=28); an approach as described above which aims for (objective) minimisation of the match between theoretical and measured Ln(G/R) versus Ln(R) patterns (R and G comprise first and second colour, eg. Red and Green, signal data).

On closer examination it turns out that the modulo-x (e.g. 28) fixed pattern noise effect is not the most effective discriminant against “falsely expressed” genes. If the pin array has a size χ×y (27×28) or modulo χy (e.g. 756) cycle—which has been confirmed experimentally: The modulo 28 effect is observable as a small ripple, but there is also a repeating “wave” and “giant excursions” occur in the same parts of the cycle. Thus a more efficient way of reducing fixed pattern noise than simply rejecting “every 28^{th} event” is to reject every event with a mean noise value greater than a threshold or outside a determined range e.g. 200-550 (say) in both Channel 1 and Channel 2.

Imposing the requirement that the mean noise be less than a threshold level, say 550, reduces the number of apparently under-expressed genes above the S/N=1 level of ˜8 bits, almost to zero. Imposing the condition that the mean noise level should not fall below a threshold level, e.g. 200 has a similar effect on the over expressed genes. No doubt many other effective alternatives will occur to the skilled person. In particular application of the techniques we describe are not limited to use with STJ-type detectors.

The techniques we describe may also be applied to: correcting fluorescence images from comparative biochemistry carried out in microtitre plates, including experiments involving whole or live cells, for example for high throughput drug discovery. Also to fluorescence imaging of whole or live cells or synthetic particles with fluorophore tagged moieties attached in biological experiments involving flow cytometry. Also to tissue and whole cell imaging by fluorescence microscopy or confocal microscopy down to the level of single molecule detection, particularly for example for the identification of the very earliest stages of the development of cancers. The spatial location and movement of individual proteins in whole cells is under early development for both basic biomedical research and for drug discovery. Most whole cell experiments are currently qualitative in nature, but there will be an increasing demand for quantitative imaging, which will require understanding and correction for coupling and energy transfer between fluorophores. Similarly in an important area of future research, at present in its infancy, which involves the characterisation of the autofluorescence of biomarkers, particularly in whole cells.

It will be understood that the invention is not limited to the described embodiments and encompasses modifications apparent to those skilled in the art lying within the spirit and scope of the claims appended hereto.

1. A method of determining respective first and second degree-of-labelling signals for different respective first and second fluorophores associated with a common entity, the method comprising:
determining a first fluorescence signal from said first and second fluorophores under first conditions;
determining a second fluorescence signal from said first and second fluorophores under second conditions different to said first conditions; and
determining said first and second degree-of-labelling signals for said first and second fluorophores from said first and second fluorescence signals; and
wherein said determining of said first and second degree-of-labelling signals is responsive to at least one coupling value (c₁₂; c₂₁) representing a coupling of energy between said fluorophores.

2. A method as claimed in claim 1 wherein said at least one coupling value represents a coupling between light emitted by one of said fluorophores and absorbed by the other of said fluorophores.

3. A method as claimed in claim 2 wherein said first fluorophore has an emission peak at a longer wavelength than said second fluorophore, and wherein said at least one coupling value represents a coupling between light emitted by said second fluorophore and absorbed by said first fluorophore.

4. A method as claimed in claim 1, wherein said first and second conditions define one or both of different illumination wavelengths and different detection wavelengths for said first and second fluorescence signal determining.

5. A method as claimed in claim 1 further comprising performing a calibration over a range of combinations of said first and second fluorophores in different proportions to determine an estimate for said at least one coupling value.

6. A method as claimed in claim 1 wherein said determining of said first and second degree-of-labelling signals is further responsive to first and second parameters (b₁; b₂) dependent on a respective degree of self-quenching for said first and second fluorophores.

7. A method as claimed in claim 1 wherein said determining of said first and second fluorescence signals comprises detecting said fluorescence signals using a superconducting tunnel junction device.

8. A method as claimed in claim 1 wherein said common entity comprises a probe entity.

9. A method as claimed in claim 1 wherein said common entity comprises a target entity.

10. A method of processing fluorescence data from a microarray according to the method of claim 1.

11. A carrier carrying processor control code to, when running, implement the method of claim 1.

12. Apparatus for determining respective first and second degree-of-labelling signals for different respective first and second fluorophores associated with a common entity, the apparatus comprising:
means for determining a first fluorescence signal from said first and second fluorophores under first conditions;
means for determining a second fluorescence signal from said first and second fluorophores under second conditions different to said first conditions;
means for determining said first and second degree-of-labelling signals for said first and second fluorophores from said first and second fluorescence signals; and
wherein said means for determining of said first and second degree-of-labelling signals is responsive to at least one coupling value (c₁₂; c₂₁) representing a coupling of energy between said fluorophores.

13. Microarray scanning apparatus incorporating the apparatus of claim 12.

14. A method of labelling an entity with a fluorophore, the method comprising
inputting a first parameter dependent on a light creation efficiency of said fluorophore;
inputting a second parameter dependent on a degree of self-quenching of said fluorophore;
determining an estimate of an optimum degree-of-labelling of said entity by said fluorophore using said first and second parameters; and
labelling said entity with said fluorophore in accordance with said estimated optimum degree-of-labelling.

15. A method as claimed in claim 14 wherein said first parameter is further dependent on one or more of a geometry of said entity, a geometry of said fluorophore, and a bonding between said fluorophore and said entity.

16. A method of labelling an entity with a plurality of fluorophores using the method of claim 14 wherein said determining of an estimate determines an estimate of an optimum degree-of-labelling of said entity by said plurality of fluorophores, and wherein said determining of said estimate is responsive to a third parameter dependent on a degree of coupling between at least two of said plurality of fluorophores.

17. A method as claimed in claim 16 further comprising performing a calibration using entities labelled with a range of different respective combinations of said plurality of fluorophores to estimate said degree of coupling.

18. An entity labelled with one or more fluorophores according to the method of claim 14.

19. An entity labelled with a plurality of different fluorophores, respective numbers of said different fluorophores being such that a fluorescence signal (S(n)) from each said fluorophore is substantially maximised.

20. A kit of fluorophore labelled probes, wherein respective numbers of said different fluorophores are such that a fluorescence signal (S(n)) from each said fluorophore is substantially maximised.

21. A method of manufacturing a kit of fluorophore labelled probes, the method comprising:
determining a combination of fluorophores for said kit; and
manufacturing said kit using said determined combination of fluorophores; and
wherein said determining of said combination of fluorophores comprises:
selecting one or both of a set of fluorophores for said kit of fluorophore labelled probes and a degree of labelling of said probes by said fluorophores using a fluorescence brightness figure of merit function (R) for a candidate said fluorophore of the set, said function comprising a function of i) a parameter dependent on a light creation efficiency of fluorophore, and ii) a parameter depending on a degree of self-quenching of the fluorophore, said selecting further being dependent on iii) a parameter dependent on a degree of coupling between the fluorophore and another fluorophore.

22-36. (canceled)

37. A method as claimed in claim 21 wherein said kit of fluorophore labelled probes is for calibration of a microarray.

38. A method as claimed in claim 21 wherein said kit of fluorophore labelled probes is for an STJ detector.

39. A kit of fluorophore labelled probes manufactured according to the method of claim 21.

40. A kit of fluorophore labelled probes comprising a kit of fluorophores, and wherein one or both of a set of fluorophores for said kit of fluorophore labelled probes and a degree-of-labelling of said probes by said fluorophores are selected using a fluorescence brightness figure of merit function (R) for a candidate said fluorophore of the set, said function comprising a function of i) a parameter dependent on a light creation efficiency of fluorophore, and ii) a parameter depending on a degree of self-quenching of the fluorophore, said selecting further being dependent on iii) a parameter dependent on a degree of coupling between the fluorophore and another fluorophore.
